(19) United States
(12) Patent Application Publication
(10) Pub. No.: US 2009/0028916 A1
(43) Pub. Date: Jan. 29, 2009

(54) Loaded Microspheres

(75) Inventors: Josep-Lluis Viladot Petit, Barcelona (ES); Gabriela Caldero-Linnhoff, Barcelona (ES)

Correspondence Address: FOX ROTHSCHILD LLP, 1101 MARKET STREET, PHILADELPHIA, PA, 19107, US

(73) Assignee: COGNIS IP MANAGEMENT GMBH, Dusseldorf (DE)

(21) Appl. No.: 12/240,860

(22) Filed: Sep. 29, 2008

Related U.S. Application Data
(62) Division of application No. 11/135,740, filed on May 24, 2005.

(30) Foreign Application Priority Data
May 25, 2004 (EP) 04012285.5

(51) Int. Cl. A61K 9/14; A61K 36/84

(52) U.S. Cl. 424/402; 424/501; 424/733

(57) ABSTRACT

The invention relates to loaded microspheres with a mean diameter of 0.001 to 1 mm, and loaded with one or more absorbed or adsorbed active principles, which are obtained by mixing fine-particle polymethyl methacrylate or methyl methacrylate cross polymer with lipophilic or hydrophilic active principles until they have been absorbed or adsorbed by the surface of the polymer and a dry powder, granules or a paste is obtained. When applied to textile or fiber finishing, the loaded microspheres provide extended release of active cosmetic or pharmaceutical active principles.

### CROSS-REFERENCE TO RELATED APPLICATIONS

This application claims priority from European Patent Application No. 04012285.5, filed on May 25, 2004.

### BACKGROUND OF THE INVENTION

“Wear comfort” is a generic term for increased demands on the part of consumers who no longer are simply content for underwear worn in direct contact with the skin, such as sheer lingerie or pantyhose for example, not to scratch or redden the skin, but—quite to the contrary—expect such underwear to have a positive effect on the condition of their skin both by helping to overcome fatigue and by imparting a fresh fragrance or avoiding roughness of the skin.

Accordingly, there has been no shortage of attempts to finish textiles and especially ladies' pantyhose—which seems to be a particularly attractive consumer field—with cosmetic active principles which are transferred to the skin during wear and produce the desired effects thereon. Now, it is obvious that the desired effects are only developed when the corresponding active principle is transferred from the carrier to the skin, i.e. after a more or less long wear time, no more active principle is present on the article of clothing. This imposes certain demands on the manufacturer of such products when it comes to the choice of the active principles because, when weighing up performance, the quantity which can be applied and, not least, the costs involved, he has to find a compromise that will lead to a product whose effect can be experienced and which the customer is able to afford despite the increase in price. Since cosmetic active principles with the desired effects are generally expensive and since additional costs are involved in the finishing of the end products, it is particularly important to the manufacturer that—apart from by contact between the finished end product and the skin of the wearer—there should be no other unwanted losses of active principles, because this would mean that the additional wear comfort expensively paid for by the consumer would be effective for only a relatively short time. A particularly unwanted form of active principle loss occurs in connection with the washing of the fibers and textiles thus finished. Even if such losses cannot be completely avoided, it is obvious that manufacturers of corresponding products are particularly concerned with applying the active principles to the fibers in such a way that they cannot be readily dissolved or mechanically detached.

Accordingly, instead of the widely practised impregnation processes, in which the active principles are directly applied to the fibers or textiles, the use of microencapsulated active principles has acquired increasing significance in recent years. Behind this is the idea of enclosing water-soluble or water-dispersible active principles in water-insoluble capsules which release the active principles during wear either by controlled release through membrane pores or by mechanical destruction of the membranes. In this way, the losses occurring over many wash cycles can actually be significantly reduced by comparison with the use of non-encapsulated active principles. Overall, however, the results thus obtained have long been unsatisfactory because the encapsulated active principles are only loosely held between the fiber fibrils and, accordingly, can easily be washed out, for example, by mechanical action during the washing process and/or require the additional use of organic binders for adhesion. Another disadvantage is that many of the known microcapsules are sufficiently stable in a watery environment, but collapse in the event of water loss and release the active principle spontaneously and uncontrollably.

Reference is made in this connection to British patent application GB 356386 A1 (Tagra) which describes a process for the production of microcapsules where a first aqueous/organic preparation of an emulsifier is mixed with a second solution or dispersion of an active principle and a polymer (for example PMMA) in an organic solvent. US 20030112491 (E Ink Corp.) describes particles, for example based on PMMA, encapsulating a liquid dye suspended in a liquid which are suitable for the production of LCD screens. EP 0611253 B1 (Novartis) relates to the encapsulation of pesticides in a system of polyisocyanates and polyamines, the reaction being carried out in the presence of nonionic polymers which may contain PMMA blocks. Finally, WO 97/023194 A1 (Cheil Jedang Corp.) describes shampoos containing microcapsules which accommodate PMMA/decadiene cross polymers besides oils.

Accordingly, the problem addressed by the present invention was to bind active principles to dermocosmetically and toxicologically safe carriers in such a way that loaded particles from which the active principle is released with delay, even in the dry state, could be produced with minimal technical complexity. A particular additional problem addressed by the invention was to produce particles with such a small mean diameter, preferably well below 1 mm, that they would be held between the fibers even without the use of binders during textile finishing.

### BRIEF SUMMARY OF THE INVENTION

The present invention relates to loaded microspheres with a mean diameter of 0.001 to 1 mm and preferably 0.01 to 0.05 mm, and loaded with one or more absorbed or adsorbed active principles, which are obtained by mixing fine-particle polymethyl methacrylate or methyl methacrylate cross polymer with lipophilic or hydrophilic active principles until they have been absorbed or adsorbed by the surface of the polymer and a dry powder, granules or a paste is obtained.

### DETAILED DESCRIPTION OF THE INVENTION

It has surprisingly been found that, among the polymers, fine-particle polymethyl methacrylate (PMMA) and especially methyl methacrylate cross polymer has an extremely high adsorption capacity for a number of active principles. The loaded microspheres show hardly any tendency to aggregate in water and are dimensionally stable, particularly after drying, i.e. do not in fact release the active principle spontaneously with the loss of water, but only with a time delay. This property may be used in particular for finishing textile materials because PMMA microspheres are so small that they become lodged between the individual fibers, even in the absence of binders, and as a result are only slowly washed out. Another advantage lies in their technically simple production which, in particular, does not involve the use of organic solvents.

The present invention also relates to a process for the production of loaded microspheres with a mean diameter of 0.001 to 1 mm and preferably 0.01 to 0.05 mm, in which fine-particle polymethyl methacrylate or methyl methacrylate cross polymer is mixed with lipophilic or hydrophilic active principles until they have been absorbed or adsorbed by the surface of the polymer and a dry powder, granules or a paste is obtained.

### Active Principles

Basically, the choice of the active principles is not critical and is determined solely by the effect to be produced on the skin. Preferred active principles for the finishing of textiles and also for cosmetic applications are those which have moisturizing properties, counteract cellulitis and/or are self-tanning. Typical examples are tocopherol, tocopherol acetate, tocopherol palmitate, carotenes, caffeine, ascorbic acid, (deoxy)ribonucleic acid and fragmentation products thereof, β-glucans, retinol, bisabolol, allantoin, phytantriol, panthenol, AHA acids, amino acids, ceramides, pseudoceramides, chitosan, dihydroxyacetone, menthol, squalane, essential oils (for example jojoba oil), vegetable proteins and hydrolysis products thereof, plant extracts, such as for example extracts of Ginkgo biloba, Camellia sinensis, Trifolium pratensis, Oleacea europensis, Lftchi sinensis, Valeriana oficinalis, Medicago sativa, Vitis vinfera, Passiflora incarnata and the like, and vitamin complexes. It is particularly preferred to use

vegetable oils, more particularly squalane.

chitosan,

menthol,

retinol (vitamin A),

caffeine,

vegetable or animal proteins and hydrolysis products thereof,

carotenes and

jojoba oil

because they have a very broad property spectrum and, individually or in combination,

contribute towards the equilibrium of the cutaneous hydrolipid layer,

prevent water loss and hence wrinkling,

freshen the skin and counteract signs of fatigue,

give the skin a soft and elastic feel,

improve dermal drainage, the supply of nutrients and the circulation,

act against oxidative stress, environmental toxins, ageing of the skin and free radicals,

compensate for the loss of fats caused by water and sun,

improve the water resistance of UV filters,

guarantee uniform tanning and, finally,

show antimicrobial properties.

Active principles predominantly suitable for the home care field are silicone oils and also surfactants, especially surfactants with a conditioning effect, such as esterquats for example. The ratio by weight between the fine-particle PMMA and the active principles is normally 10:90 to 99:1 and preferably 75:25 to 90:10.

### Production of the Microspheres

To produce the microspheres, the fine-particle PMMA and the active principles are first mixed together. The active principles are preferably present as liquids or as aqueous or alcoholic solutions or dispersions. The PMMA has a high absorption capacity for liquids so that pastes, granules and, in particular, dry powders are obtained after mixing. The active principles are not just deposited onto the polymer particles, but above all are physically adsorbed. If this were not the case, there would be no delayed release in practice, just spontaneous release. Thereafter, it is advisable to disperse the loaded particles with intensive shearing in an aqueous or oil-containing phase which may optionally contain other additives such as, for example, thickeners and/or preservatives. Under a microscope, it can be seen that the particles are discrete with a mean diameter of well below 1 mm and preferably in the range from 0.01 to 0.1 mm.

### Commercial Applications

The present invention relates to the use of the new loaded microspheres in various fields, i.e.

for the finishing of fibers and textile materials, more particularly for the conditioning thereof;

for the production of cosmetic and/or pharmaceutical preparations;

for the production of laundry detergents, dishwashing detergents and cleaning compositions;

for the production of food supplements;

for the incorporation of additives in paper, paints and coatings.

### EXAMPLES

### Example 1

### Production of Microspheres for the Finishing of Textiles and Fibers

In a 100 ml three-necked flask, 5 g perfume oil and 10 g PMMA microspheres (Covabead® LH 85, a product of LCW) were mixed by slow stirring until the active principle had been completely adsorbed by the surface of the polymer and a dry powder was present. The powder was then stirred with intensive shearing into a solution of 1 g thickener (Carbopol® ETD 2020) and 0.5 g preservative (Phenonip®) in 85 ml water. Under a microscope, it could be seen that discrete microspheres with a mean diameter of 0.01 mm were present.

### Example 2

### Production of Microspheres for the Finishing of Textiles and Fibers

In a 100 ml three-necked flask, 10 ml of a 10% by weight ethanolic solution of menthol and 10 g PMMA microspheres (Covabead® LH 85, a product of LCW) were mixed by slow stirring until the active principle had been completely adsorbed by the surface of the polymer and a dry powder was present. The powder was then stirred with intensive shearing into a solution of 1 g thickener (Carbopol® ETD 2020) and 0.5 g preservative (Phenonip®) in 80 ml water. Under a microscope, it could be seen that discrete microspheres with a mean diameter of 0.01 mm were present.

### Example 3

### Production of Microspheres for the Food Supplements Field

In a 100 ml three-necked flask, 7 g of an aqueous preparation of an extract of Valediana oficinalis with an active principle content of 25% by weight and 15 g PMMA microspheres (Covabead® LH 85, a product of LCW) were mixed by slow stirring until the active principle had been completely adsorbed by the surface of the polymer and a dry powder was present. The powder was then stirred with intensive shearing into a solution of 1 g thickener (Carbopol® ETD 2020) and 0.5 g preservative (Phenonip®) in 78 ml water. Under a microscope, it could be seen that discrete microspheres with a mean diameter of 0.01 mm were present.

### Example 4

### Production of Microspheres for the Cosmetics Field

In a 100 ml three-necked flask, 7 g of a 15% by weight dispersion of β-carotene in almond oil and 15 g PMMA microspheres (Covabead® LH 85, a product of LCW) were mixed by slow stirring until the active principle had been completely adsorbed by the surface of the polymer and a dry powder was present. The powder was then stirred with intensive shearing into a solution of 1 g thickener (Carbopol® ETD 2020) and 0.5 g preservative (Phenonip®) in 78 ml water. Under a microscope, it could be seen that discrete microspheres with a mean diameter of 0.01 mm were present.

### Example 5

### Production of Microspheres for the Home Care Field

In a 100 ml three-necked flask, 7 g of a 15% by weight solution of an esterquat (Dehyquart® AU 46) in isopropyl alcohol and 15 g PMMA microspheres (Covabead® LH 85, a product of LCW) were mixed by slow stirring until the active principle had been completely adsorbed by the surface of the polymer and a dry powder was present. The powder was then stirred with intensive shearing into a solution of 1 g thickener (Carbopol® ETD 2020) and 0.5 g preservative (Phenonip®) in 78 ml water. Under a microscope, it could be seen that discrete microspheres with a mean diameter of 0.01 mm were present.

What is claimed is:

1. Loaded microspheres with a mean diameter of 0.001 to 1 mm obtainable by mixing fine-particle polymethyl methacrylate or methyl methacrylate cross polymer with one or more lipophilic or hydrophilic active principles until the one or more active principles have been absorbed or adsorbed by the surface of the polymers and a dry powder, granules or paste is obtained.

2. Loaded particles according to claim 1, wherein the polymer with the one or more absorbed or adsorbed active principles is obtained as a dry powder.

3. Loaded particles according to claim 1, wherein the obtained dry powder, granules or paste microspheres are dispersed with intensive shearing in an aqueous or oil-containing medium, optionally in the presence of other additives.

4. Loaded microspheres according to claim 1, with a mean diameter of 0.01 to 0.1 mm.

5. Loaded microspheres according to claim 1, wherein the one or more active principles are selected from the group consisting of tocopherol, tocopherol acetate, tocopherol palmitate, carotenes, caffeine, ascorbic acid, (deoxy)ribonucleic acid and fragmentation products thereof, β-glucans, retinol, bisabolol, allantoin, phytantriol, panthenol, AHA acids, amino acids, ceramides, pseudoceramides, chitosan, dihydroxyacetone, menthol, squalane, essential oils, vegetable or animal proteins and hydrolysis products thereof, plant extracts, silicone oils and surfactants.

6. Loaded microspheres according to claim 1, wherein the fine-particle polymethyl methacrylate or methyl methacrylate cross polymer and the one or more active principles are used in a ratio by weight of 10:90 to 99:1.

7. Loaded microspheres according to claim 1, wherein the fine-particle polymethyl methacrylate or methyl methacrylate cross polymer and the one or more active principles are used in a ratio by weight of 75:25 to 90:10.

8. A process for the production of loaded microspheres with a mean diameter of 0.001 to 1 mm, said process comprising mixing fine-particle polymethyl methacrylate or methyl methacrylate cross polymer with one or more lipophilic or hydrophilic active principles until the one or more active principles have been absorbed or adsorbed by the surface of the polymers and a dry powder, granules or paste is obtained.

9. The process according to claim 8, wherein the obtained dry powder, granules or paste microspheres are dispersed with intensive shearing in an aqueous or oil-containing medium, optionally in the presence of other additives.

10. The process according to claim 9, wherein the other optional additives include one or more thickeners or preservatives.

11. The process according to claim 8, wherein the fine-particle polymethyl methacrylate or methyl methacrylate cross polymer and the one or more active principles are used in a ratio by weight of 10:90 to 99:1.

12. The process according to claim 8, wherein the fine-particle polymethyl methacrylate or methyl methacrylate cross polymer and the one or more active principles are used in a ratio by weight of 75:25 to 90:10.

13. The process according to claim 8, wherein the one or more active principles are present for mixing as liquids or as aqueous or alcoholic solutions or dispersions.

14. A process according to claim 8, wherein the one or more active principles are selected from the group consisting of tocopherol, tocopherol acetate, tocopherol palmitate, carotenes, caffeine, ascorbic acid, (deoxy)ribonucleic acid and fragmentation products thereof, β-glucans, retinol, bisabolol, allantoin, phytantriol, panthenol, AHA acids, amino acids, ceramides, pseudoceramides, chitosan, dihydroxyacetone, menthol, squalane, essential oils, vegetable or animal proteins and hydrolysis products thereof, plant extracts, silicone oils and surfactants.

15. The process according to claim 8, wherein the loaded microspheres have a mean diameter of 0.01 to 0.1 mm.

16. A process for adapting textiles or fibrous materials to provide extended release of cosmetically or pharmaceutically active principles, said process comprising embedding in such textiles or fibrous materials loaded microspheres with a mean diameter of 0.001 to 1 mm obtained by mixing fine-particle polymethyl methacrylate or methyl methacrylate cross polymer with one or more lipophilic or hydrophilic active principles until the one or more active principles have been absorbed or adsorbed by the surface of the polymers and a dry powder, granules or paste is obtained.

17. The process according to claim 16, wherein the obtained dry powder, granules or paste microspheres are further treated by dispersing them by intensive shearing in an aqueous or oil-containing medium, optionally in the presence of other additives.

18. The process according to claim 17, wherein the other optional additives include one or more thickeners or preservatives.

19. The process according to claim 16, wherein the loaded microspheres have a mean diameter of 0.01 to 0.1 mm.

20. The process according to claim 16, wherein the one or more active principles are selected from the group consisting of tocopherol, tocopherol acetate, tocopherol palmitate, carotenes, caffeine, ascorbic acid, (deoxy)ribonucleic acid and fragmentation products thereof, β-glucans, retinol, bisabolol, allantoin, phytantriol, panthenol, AHA acids, amino acids, ceramides, pseudoceramides, chitosan, dihydroxyacetone, menthol, squalane, essential oils, vegetable or animal proteins and hydrolysis products thereof, plant extracts, silicone oils and surfactants.
